(19) United States
(12) Patent Application Publication
(10) Pub. No.: US 2004/0229826 A1
(43) Pub. Date: Nov. 18, 2004

(54) Dose-dense & sequential adjuvant cancer chemotherapy

(76) Inventor: Larry Norton, New York, NY (US)

Correspondence Address: OPPEDAHL AND LARSON LLP, P O BOX 5068, DILLON, CO, 80435-5068, US

(21) Appl. No.: 10/735,180

(22) Filed: Dec. 12, 2003

Related U.S. Application Data
(60) Provisional application No. 60/432,840, filed on Dec. 12, 2002.

(51) Int. Cl. A61K 31/704; A61K 31/66; A61K 31/337

(52) U.S. Cl. 514/34; 514/109; 514/449

(57) ABSTRACT

Breast cancer is treated by (a) administering to a patient in a first plurality of chemotherapy cycles a therapeutically-effective and well-tolerated amount of doxorubicin in a dose-dense protocol; (b) subsequently administering to the patient in a second plurality of chemotherapy cycles a therapeutically-effective and well-tolerated amount of a taxane chemotherapy agent, for example paclitaxel, in a dose-dense protocol; and (c) subsequently administering to the patient in a third plurality of chemotherapy cycles a therapeutically-effective and well-tolerated amount of cyclophosphainide in a dose-dense protocol. Preferably, the dose dense interval between treatments is about 14 days. The number of cycles in each plurality of chemotherapy cycles is suitably 3 or more, preferably 4. Suitable well-tolerated treatment levels are 60 mg/m² of doxorubicin, 175 mg/² of paclitaxel, and 600 mg/² of cyclophoshamide. A therapeutically effective amount of G-CSF may also be administered during the intervals between treatments in one or more of the chemotherapy cycles.

This application claims the benefit under 35 USC § 19(e) of U.S. Provisional Application Dec. 12, 2003, filed Dec. 12, 2002, which application is incorporated herein by reference in its entirety.

### BACKGROUND OF THE INVENTION

The present invention relates to a method of administering chemotherapeutic agents where the drugs are administered to patients suffering from cancer in a specified sequence with a shortened inter-treatment interval in order to improve killing of the cancer cells and the survival of the patients. The invention is exemplified with reference to breast cancer.

Effective cancer chemotherapy represents a balance between the toxicity of the administered therapeutic agent to the cancer cells and the toxicity of the therapeutic agent to the patient. To maximize the former, the dosage used is frequently the maximum tolerable dose, which can lead to substantial side effects, and reduce quality of life. It is therefore desirable to identify treatment protocols that provide effective anti-cancer results with the minimum side effects.

Theoretical evaluations and mathematical modeling of the mechanics of cancer chemotherapy have reported over the years, and these considerations suggest that dose-dense protocols may be able to provide effective cancer therapy. The concept, however, was only theoretical, and heretofore has not been proven through controlled study of actual patients, to actually provide the predicted results. Furthermore, providing more frequent doses of toxic agents seems antithetical to attaining the goal of minimization of side effects.

Advances in the adjuvant chemotherapy of primary, operable breast cancer have come both from the introduction of effective agents and from the application of the principles of combination chemotherapy, which underlie much of contemporary oncology. (Fisher B. From Halsted to prevention and beyond: Advances in the management of breast cancer during the twentieth century. Eur J Cancer 35:1963-1973, 1999; DeVita V T Jr, Young R C, Canellos G P. Combination versus single agent chemotherapy: a review of the basis for selection of drug treatment of cancer. Cancer 35:98-110, 1975) Attempts to advance those principles in the treatment of breast cancer by substantial escalation of drug dosage levels have so far proven unsuccessful. (Peters W P, Rosner G, Vredenburgh J, et al: Updated results of a prospective, randomized comparison of two doses of combination alkyating agents as consolidation after CAF in high-risk primary breast cancer involving ten or more axillary lymph nodes: CALGB 9082/SWOG 9114/NCIC MA-13. Proc Am Soc Clin Oncol 81, 2001 (abstr); Crown J P, Lind M, Gould A, et al: High-dose chemotherapy with autograft support is not superior to cyclophosphamide, methotrexate and 5-FU following doxorubicin induction in patients with breast cancer and four or more involved axillary lymph nodes. Proc Am Soc Clin Oncol 166, 2002 (abstr)) Indeed, for the three most useful agents, doxorubicin (A for Adriamycin®), cyclophosphamide (C for Cytoxan®), and paclitaxel (T for Taxol®), dose levels higher than 60 mg/m², 600 mg/m², and 175 mg/m² (given over three hours) respectively, are not more effective. (Budman D R, Berry D A, Cirrincione C T, et al: Dose and dose intensity as determinants of outcome in the adjuvant treatment of breast cancer. The Cancer and Leukemia Group B. J Natl Cancer Inst 90:1205-1211, 1998; Fisher B, Anderson S, DeCillis A, et al: Further evaluation of intensified and increased total dose of cyclophosphamide for the treatment of primary breast cancer: findings from National Surgical Adjuvant Breast and Bowel Project B-25. J Clin Oncol 17:3374-3388, 1999; Winer E, Berry D, Duggan D, et al: CALGB 9342: A randomized trial of three doses of paclitaxel in patients with metastatic breast cancer. Semin Oncol 26:9, 1999.)

Hudis et al. report 5-year follow-up results of a Phase II study treating breast cancer with in high risk node-positive patients with dose-intensive sequential adjuvant therapy. J Clinical Oncology 17: 93-100 (1999). In this study, dose dense treatments with sequential doxorubicin (90 mg/m²), paclitaxel (250 mg/m²/24 hour) and cyclophosphamide (3 g/m²). The chemotherapeutic agents were each given three times, and intervals of two weeks. Between treatments, granulocyte colony stimulating factor (GCSF) was administered at a level of 5 μg/kg. The paper concludes that “dose-dense therapy using sequential doxorubicin, paclitaxel, and cyclophosphamide at escalated doses is feasible and associated with promising disease-free survival results.”

### SUMMARY OF THE INVENTION

The present invention provides a method for enhancing the efficacy of chemotherapeutic treatments making use of a combinations of therapeutic agents. In accordance with the method of the invention, improved chemotherapeutic efficacy is achieved by the steps of:

(a) administering to a cancer patient in need of chemotherapeutic treatment a first plurality of chemotherapy cycles a therapeutically-effective and well-tolerated amount of a first chemotherapy agent, said first plurality chemotherapy cycles being administered in a dose-dense protocol; and

(b) after the completion of the first plurality of chemotherapy cycles, administering to the patient in a second plurality of chemotherapy cycles a therapeutically-effective and well-tolerated amount of a second chemotherapy agent, different from the first chemotherapy agent, said second plurality of chemotherapy cycles being administered in a dose-dense protocol. Additional chemotherapy agents can be included in further sets of chemotherapy cycles where additional agents are indicated for the treatment of the specific cancer.

For example, in accordance with one specific embodiment of the invention there is provided a method for treating breast cancer in a patient having a chemotherapy responsive breast cancer, comprising the steps of:

(a) administering to the patient in a first plurality of chemotherapy cycles a therapeutically-effective and well-tolerated amount of doxorubicin, said first plurality chemotherapy cycles being administered in a dose-dense protocol;

(b) after the completion of the first plurality of chemotherapy cycles, administering to the patient in a second plurality of chemotherapy cycles a therapeutically-effective and well-tolerated amount of a taxane chemotherapy agent, said second plurality of chemotherapy cycles being administered in a dose-dense protocol; and

(c) after the completion of the second plurality of chemotherapy cycles, administering to the patient in a third plurality of chemotherapy cycles a therapeutically-effective and well-tolerated amount of cyclophosphamide, said third plurality of chemotherapy cycles being administered in a dose-dense protocol. Preferably, the dose dense interval between treatments in this embodiment is about 14 days. The number of cycles in each plurality of chemotherapy cycles is suitably 3 or more, preferably 4. Suitable treatment levels are 60 mg/m² of doxorubicin, 175 mg/m² of paclitaxel, and 600 mg/m² of cyclophoshamide. It is noted that these dosages are substantially lower than the levels reported by Hudis et al, supra, and therefore that less toxicity to the patient results. As demonstrated below, however, comparable efficacy against the cancer is observed.

In a further embodiment of the invention, including as an embodiment of the mentioned embodiments for the treatment of breast cancer, the method further comprises the step of administering to the patient a therapeutically effective amount of G-CSF during the intervals between treatments in one or more of the chemotherapy cycles.

### BRIEF DESCRIPTION OF THE FIGURES

FIG. 1 shows the various treatment regimens tested.

FIG. 2a. shows Disease-Free Survival by Dose Density

FIG. 2b shows Overall Survival by Dose Density

FIG. 3a. shows Disease-Free Survival by Sequence

FIG. 3b shows Overall Survival by Sequence

FIG. 4a. shows Disease-Free Survival by Treatment Arm

FIG. 4b shows Overall Survival by Treatment Arm

### DETAILED DESCRIPTION OF THE INVENTION

The present invention is based on initial results of a prospective, randomized study coordinated by the Cancer and Leukemia Group B on behalf of the National Cancer Institute's Breast Intergroup, INT C9741. This study tested two novel concepts, based on experimental data and mathematical reasoning. These concepts, dose density and sequential therapy, build upon and further develop the theory of combination chemotherapy. (Norton L. Theoretical concepts and the emerging role of taxanes in adjuvant therapy. The Oncologist 3:30-35, 2001. ) The results of the study establish that dose dense, sequential adjuvant therapy provides effective cancer treatment.

Dose density refers to the administration of drugs with a shortened inter-treatment interval. It is based on the observation that in experimental models a given dose always kills a certain fraction, rather than a certain number, of exponentially-growing cancer cells. (Skipper H E. Laboratory models: some historical perspectives. Cancer Treat Rep 70:3-7, 1986.) Since human cancers in general, and breast cancers in particular, usually grow by non-exponential Gompertzian kinetics, this model has been extended to those situations.(Norton L, Simon R, Brereton J D, Bogden A E. Predicting the course of Gompertzian growth. Nature 264:542-545, 1976.; Norton L, Simon R. The growth curve of an experimental solid tumor following radiotherapy. J Natl Cancer Inst 58:1735-1741, 1977; Norton L, Simon R. Tumor size, sensitivity to therapy and the design of treatment protocols. Cancer Treat Rep 61:1307-1317, 1977; Norton L. A Gompertzian model of human breast cancer growth. Cancer Res 48:7067-7071, 1988; Norton L. Implications of kinetic heterogeneity in clinical oncology. Semin Oncol 12:231-249, 1985.) Regrowth of cancer cells between cycles of cyto-reduction is more rapid in volume-reduced Gompertzian cancer models than in exponential models. Hence, it has been hypothesized that the more frequent administration of cytotoxic therapy would be a more effective way of minimizing residual tumor burden than mere dose escalation. In the INT C9741 trial, the dose dense schedule is accomplished by using granulocyte colony stimulating factor (G-CSF or filgrastim or Neupogen®) to reduce side effects and permit dose-dense cycling of the drugs A, T, and C at their optimal dose levels rather than at the conventional three-week intervals.

As used in the specification and claims of this application, the term “dose-dense protocol” refers to the use of an interval between treatments which is sufficiently short to preclude the complete regrowth of cancer cells to levels equivalent to those present before the treatment. In the case of the treatment for breast cancer as described below, this period is about 14 days. It will be appreciated that some flexibility exists in this number to allow for scheduling issues, for example intervals of 12-16 days. However, the dose dense protocols of the invention for treatment of breast cancer do not encompass inter-treatment intervals as long as 21 days.

It will be appreciated that shorter intervals may lead to increased toxicity to the patient. Thus, an alternative way to look at dose density is to first find the lowest dose level of a particular drug that kills the most cancer cells and giving the drug at this dose level as often as possible. For example, clinical research demonstrated that 30 mg/m² of doxorubicin is inferior in treating breast cancer to 40 mg/m² and 60 mg/m². (Wood W C, Budman D R, Korzun A H, Cooper M R, Younger J, Hart R D, Moore A, Ellerton J A, Norton L, Ferree C R, Ballow A C, Frei E, III, Henderson I C. Dose and Dose Intensity Trial of Adjuvant Chemotherapy for Stage II, Node-Positive Breast Carcinoma. [published erratum appears in N Engl J Med 331:139, 1994]. New Engl J Med 330:1253-1259, 1994.) A later trial proved that doses of 75 mg/m² and 90 mg/m² do not kill more breast cancer cells, but does cause more toxicity. (Henderson I C, Berry D A, Demetri G D, Cirrincione C T, Goldstein L J, Martino S, Ingle J N, Cooper M R, Hayes D F, Tkaczuk K H, Fleming G, Holland J F, Duggan D B, Carpenter J T, Frei E 3rd, Schilsky R L, Wood W C, Muss H B, Norton L. Improved outcomes from adding sequential Paclitaxel but not from escalating Doxorubicin dose in an adjuvant chemotherapy regimen for patients with node-positive primary breast cancer. J Clin Oncol. 2003 Mar 15;21(6):976-83.) Hence, the “optimal” dose level is 60 mg/m². Parallel research determined that 175 mg/m² of paclitaxel kills more breast cancer cells that lower dose levels but higher dose levels are not more effective. (Winer E., Berry D., Duggan D., Henderson I. C., Cirrincione C., Cooper M. R., Norton L. CALGB 9342: A randomized trial of three doses of paclitaxel in patients with metastatic breast cancer. Semin Oncol 26:9, 1999.) Similarly, the optimal dose of cyclophosphamide is 600 mg/m². A great deal of clinical research from the early 1990s on was necessary to develop the specific regimens that were used in the definitive test of the procedure of dose density. However, these earlier trials did not include control arms and were therefore not proof of the value of the procedure.

Dose-dense therapy is demonstrated by the results of this application to be generally applicable to improvement of chemotherapeutic treatment for patients suffering from cancer. Thus, when one is administering to the patient one or more types of chemotherapeutic agent in a plurality of treatments, the results are improved by making use of the optimal amount of each type of chemotherapeutic agent and giving the treatments in a dose-dense protocol, preferably at the shortest tolerated intervals. Of note is that in this pivotal trial described below, the three drugs were used in two ways, as AC->T and as A->T->C, and both were more effective when given in the dose dense (two-week) fashion than in the conventional three-week procedure. Sequential therapy refers to the application of chemotherapeutic agents one at a time rather than concurrently. It does not challenge the concept that multiple drugs are needed to maximally perturb cancers that are comprised of cells heterogeneous in drug sensitivity. Rather, it hypothesizes that for slow-growing cancers like most breast cancers it is more important to preserve dose density than to force a combination, especially if that combination would be more toxic to the patient and requires dose-reductions or delays in drug administration. If dose density is the same in a sequential combination chemotherapy regimen and a concurrent combination regimen, theoretical considerations suggest that the therapeutic results should be the same, even if the sequential pattern happens to be less toxic.

As used in the specification and claims of this application, the phrase “therapeutically effective and well-tolerated amount” refers to an amount of the chemotherapy agent that is sufficient to provide a therapeutic benefit by reducing tumor size (in a preoperative patient) or increasing the period of disease free and/or overall survival without giving rise to substantial hematological side effects in an average patient population. In particular hematological side effects such as neutropenia and neutropenic fever. In the Hudis, et al. 1999 study using tolerable, but not well-tolerated dosages, neutropenic fever was observed in 69% of the patients, and 67% requiring transfusion of packed red blood cells. In contrast, as shown in Table 4, the overall side effect toxicities, including hematological side effects are much lower, with the well-tolerated doses of the invention. Chemotherapeutic agents have been shown to have concentrations of maximum effectiveness, above which the dose-response curve for the killing of cancer cells plateaus. This concentration represents an upper limit for the concentration of the chemotherapy agent employed in the present invention, since additional therapeutic agent increases side effects without increasing therapeutic benefit.

As used in the specification and claims of this application, the term “administering” refers to the delivery of the specified agent to the patient, regardless of the delivery method. In general, the delivery will be via an intravenous injection, which may be a short time push or a longer term infusion (for example 24 hours in the case of a taxane like paclitaxel). It will be appreciated that different individuals may deliver individual treatments during practice of the invention, and that some treatments, in particular daily G-CSF treatments may be self-administered by the patient.

The invention will now be exemplified through the results of the dose-dense, sequential adjuvant study in breast cancer patients.

### EXAMPLE

### Methods

Study Plan

This Intergroup trial, coordinated by the Cancer and Leukemia Group B (CALGB) with participation from the Eastern Cooperative Group (ECOG), Southwest Oncology Group (SWOG), and North Central Cancer Treatment Group (NCCTG), was open for patient accrual between September 1997 and March 1999. Its objective was to treat women with primary adenocarcinoma of the breast (including metaplastic and bilateral lesions) and no metastases other than histologically-involved axillary lymph nodes (T0-T3, N1-2, M0). (16) Primary therapy consisted of removal of the entire cancer by a segmental mastectomy (lumpectomy) plus axillary dissection, or a modified radical mastectomy, with no gross or microscopic invasive tumor at the resection margin. Required laboratory data were limited to an initial bilirubin level within institutional normal limits and, before each cycle of chemotherapy, including the first, a granulocyte count of at least 1000/μL and a platelet count of at least 100,000/μL. Eligible patients also had pretreatment chest radiographs and electrocardiograms. All patients provided informed consent meeting all federal, state, and institutional guidelines.

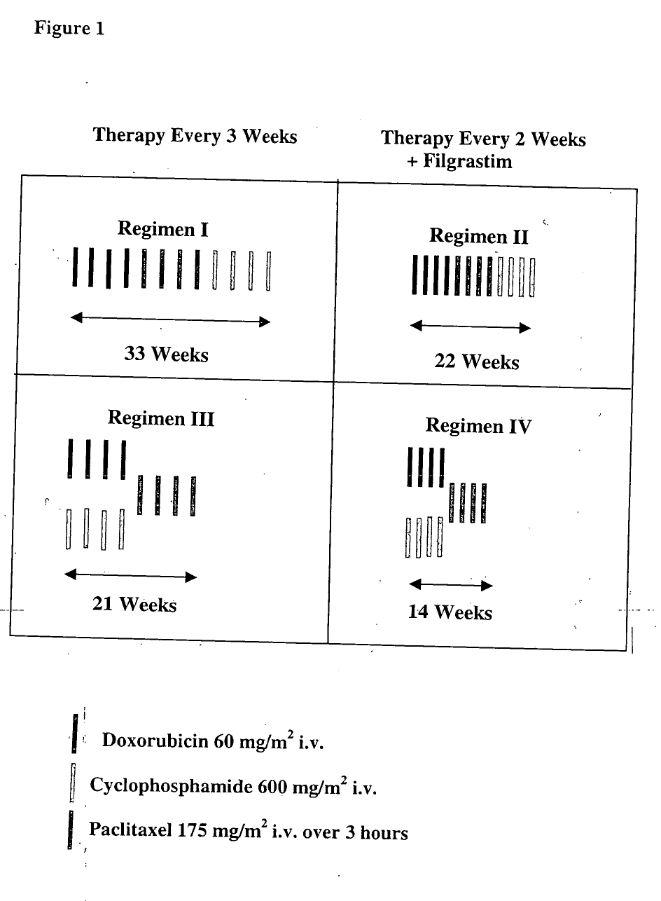

Designed for outpatients, all chemotherapy (FIG. 1) was given intravenously, starting within 84 days from primary surgery. The study used a 2×2 factorial experimental design to assess the two factors of dose density (2 weeks versus 3 weeks) and treatment sequence (concurrent versus sequential) and the possible interaction between them Patients were assigned with equal probability to one of four treatment regimens: (I) doxorubicin 60 mg/m2 every three weeks for four cycles followed by paclitaxel 175 mg/m2 every three weeks for four cycles followed by cyclophosphamide 600 mg/m2 every three weeks for four cycles; (II) doxorubicin 60 mg/m2 every two weeks for four cycles followed by paclitaxel 175 mg/m2 every two weeks for four cycles followed by cyclophosphamide 600 mg/m2 every two weeks for four cycles, filgrastim days 3-10 of each cycle at 5 μg/kg rounded to either 300 μg or 480 μg total dose; (III) doxorubicin 60 mg/m2 plus cyclophosphamide 600 mg/m2 every three weeks for four cycles followed by paclitaxel 175 mg/m2 every three weeks for four cycles; (IV) doxorubicin 60 mg/m2 plus cyclophosphamide 600 mg/m2 every two weeks for four cycles followed by paclitaxel 175 mg/m2 every two weeks for four cycles, filgrastim days 3-10 of each cycle at 5 μg/kg rounded to either 300 μg or 480 μg total dose. Regimen III was the superior arm of protocol INT C9344 in which it was compared to four cycles of AC every three weeks not followed by paclitaxel.(17) Regimen II, the most unconventional dose-schedule, being both dose-dense and sequential, had previously been piloted in concept by Hudis and colleagues.(18)

Complete blood counts were done prior to each chemotherapy treatment. If the granulocyte count was less than 1,000/μl or the platelet count less than 100,000/μl on the scheduled day, chemotherapy was delayed until those minimal levels were achieved. If there was more than a 3 week delay, the study chair was contacted. Chemotherapy dose modifications were discussed with the study chair. When modifications were indicated due to toxicity, the drug dose was lowered by 25% decrements according to the degree of toxicity.

Radiation therapy, when used, was given after the completion of chemotherapy. Although recommendations regarding technique were included in the written protocol, investigators were permitted to follow institutional guidelines. It was recommended but not required that tamoxifen 20 mg per day be started within 12 weeks after completion of chemotherapy and be given for five years to all premenopausal patients with hormone receptor-positive cancers and to all postmenopausal patients irrespective of receptor status.

Disease-free survival (DFS), which was the primary study endpoint, was measured from study entry until local recurrence, distant relapse or death without relapse, whichever occurred first. Disease spread to the opposite breast which occurred concurrently with local and/or other distant sites was considered relapse; however, disease spread to the opposite breast in the absence of local and distant recurrence was considered a second primary. All second primaries regardless of site are considered adverse events and not failures in DFS. Surviving patients who were disease-free were censored at the date last known to be free from their primary breast cancer. The secondary endpoint of overall survival (OS) was measured from study entry until death from any cause; surviving patients were censored at the date of last contact. Death due to AML/MDS was considered treatment-related. Target accrual was 1584 patients over 22 months with the study analysis at three years after completing accrual. This provided 90% power to detect a 33% difference in hazard for either main effect, assuming an event rate equal to that in an earlier Intergroup (CALGB) trial. (5) Cox proportional hazards regressions with Wald chi-square tests were used to model and assess the relation between DFS and OS, respectively, and treatment factors with clinical variables. Kaplan-Meier curves with logrank tests were used to compare the distribution of time to events. Comparisons of two or more proportions used contingency table analysis. 95% confidence intervals used exact binomial methods. All p-values are two-sided. Toxicity grading used the CALGB expanded common toxicity criteria. Patient information was collected on standard CALGB study forms by the CALGB Data Operations unit located in Durham, N.C. and entered into the official CALGB database. Data were current as of May 2002.

### Experiment

Between September, 1997 and March, 1999, 2005 volunteer female patients were accrued from CALGB (41%), ECOG (30%), SWOG (16%) and NCCTG (13%). This total was increased from that planned (1584) in an attempt to compensate for a faster than expected accrual rate. Thirty-two patients never received any protocol therapy. The 1973 patients (>98%) who were treated provide the basis for this report (Table 1). Their median age was fifty years, 65% had estrogen receptor-positive tumors, the median number of involved lymph nodes was three, and 12% had ten or more involved axillary lymph nodes. The regimens were balanced with regard to these and all other major pretreatment variables. The maximum and median follow-up times are five and three years respectively. After a median follow-up of 36 months, 315 patients had relapsed or died compared with 515 expected failures under the assumption that both arms would have the event rate we observed in CALGB 8541. The smaller number of failures than expected is partly explained by the rapid accrual rate, and partly by the more favorable course of all women in the trial compared to prior CALGB studies.

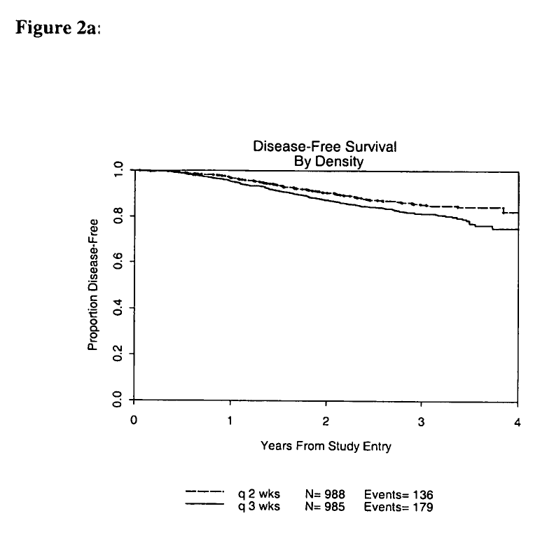

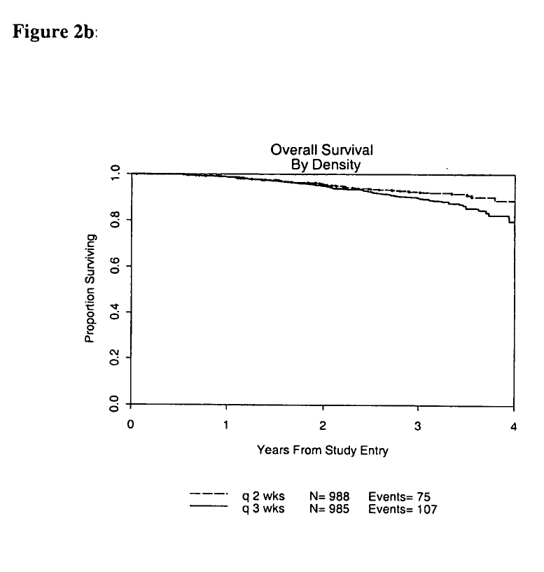

As Table 3a indicates, DFS was significantly prolonged for the dose-dense regimens (II and IV) compared with the every three-week regimens (I and III) (Risk Ratio [RR]=0.74, P=0.010). This dose density effect remained statistically significant even after adjusting for number of positive nodes, tumor size, menopausal status and tumor estrogen receptor status. Treatment sequence did not correlate with DFS (P=0.58), nor was there an interaction between dose density and treatment sequence (P=0.40). FIGS. 2a, 3a, and 4a show pictorially the main effects of dose density and treatment sequence and the lack of interaction between the two factors, respectively.

The DFS for the dose-dense and conventional three-week schedules at one year was 97% vs. 95% at two years was 91% vs. 87% at three years was 85% vs. 81%, and at four years was 82% vs. 75%. The first two of these (both the absolute figures and relative difference) will change little with further follow-up. The reason is that all patients have been in the trial for longer than two years and complete data are available for 99% of the patients at one year and 92% at two years. The relative reduction in hazard of recurrence attributed to dose-dense schedule was 28% at one year, 13% at two years, 50% at three years and 52% at four years. Although these latter estimates have large standard errors, this suggests that the benefit of dose-density continues into the period of longer follow-up.

The overall relative reduction in hazard attributed to dose-dense therapy was 19% for ER-positive and 32% for ER-negative tumors. This difference by ER status (interaction between ER and treatment) is not statistically significant.

There were no differences in the pattern of local recurrences for either treatment factor (dose-density or sequence) despite differences in time from surgery to local radiation therapy (19-37 weeks).

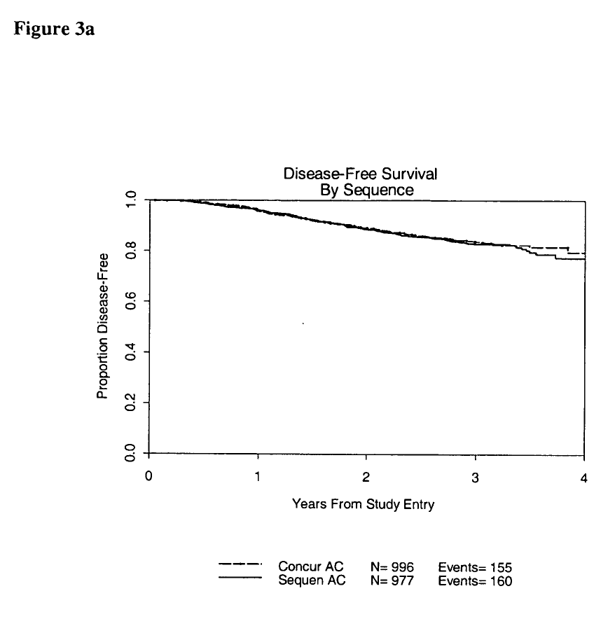

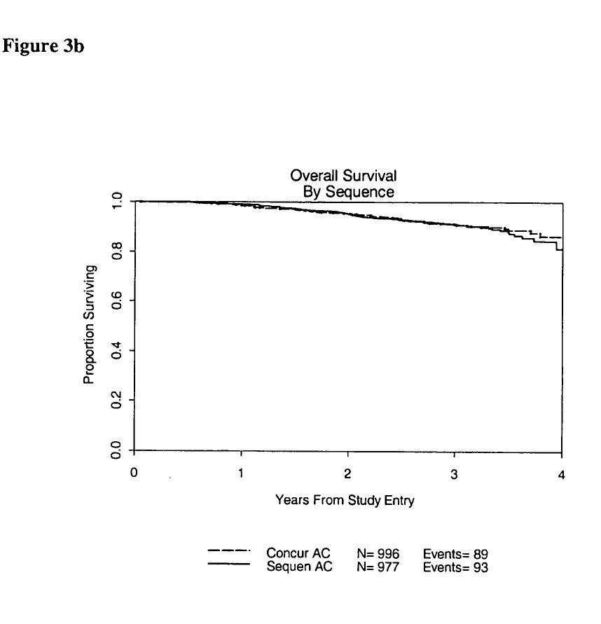

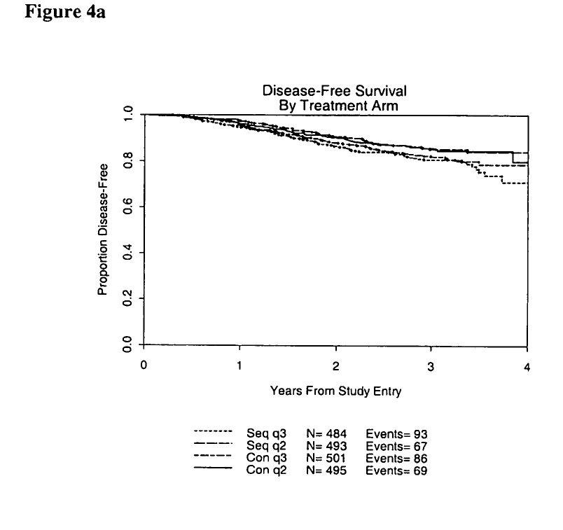

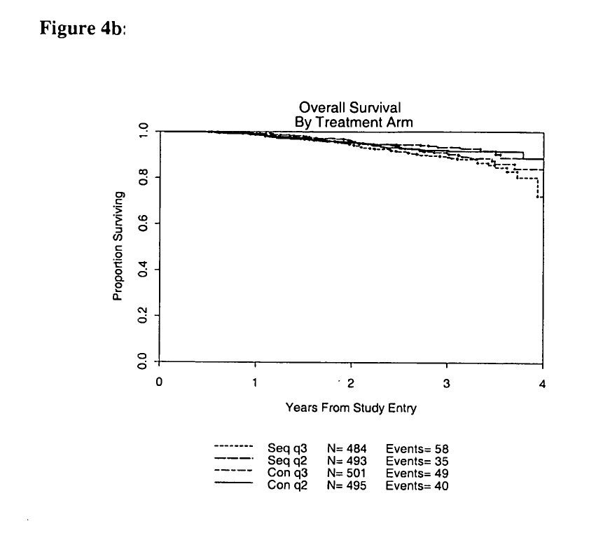

Table 3b shows that OS was significantly prolonged in the dose-dense regimens (RR=0.69, P=0.013), even after adjusting for the standard clinical pretreatment variables mentioned previously. Treatment sequence was not significantly correlated with OS P=0.48). There was no interaction between density and sequence of treatment (P=0.13). FIGS. 2b and 3b show the relation between OS and density and sequence, respectively. FIG. 4b shows the lack of interaction between the two factors.

The 3-year OS was 92% in the dose-dense regimens and 90% for those receiving 3-week treatment. Similar to DFS, the OS results out to two years will change little with further observation. However, the observed survival benefit of dose density occurs beyond two years and therefore is subject to greater change than that for DFS. On the other hand, OS benefit emerging later than DFS benefit is biologically tenable and adds credence to the observed survival benefit.

The sites of first recurrence are shown in Table 2. Although this study is not designed for formal comparisons among arms, the pattern of failure was similar among regimens.

Standard toxicity data were available for 1962 patients. Detailed toxicity and complication data were available for 412 patients over 3973 treatment cycles (Table 4). There were no treatment-related deaths during therapy. There was only one death within the first six months of protocol treatment; the cause of death, cerebral infarction, was considered unrelated to treatment. The number of cycle delays was relatively small, ranging from 7% on regimens I and II to 8% and 6% on regimens III and IV, respectively. Of the cycles delayed, 38% of the delays on the q 3 week regimens were due to hematologic toxicity compared to 15% on the q 2 week regimens (p<0.0001). Dose reductions were infrequent (Table 5). Overall only 3% of patients were hospitalized for febrile neutropenia. Grade 4 granulocytopenia (<500/μL) was more frequent on the three-week regimens compared to the dose-dense regimens (33% versus 6%, P<0.0001). While 13% of patients on the concurrent dose-dense regimen (IV) underwent at least one red blood cell transfusion, there were no transfusions on the sequential three-week treatment (I) and <4% in each of the other two regimens (P=0.0002). Grade 3 or higher emesis was significantly more common for the concurrent regimens than for the sequential regimens (7% versus 3%, P=0.0002)

There have been six treatment-related deaths (Table 6), all occurring between 23 and 41 months after the beginning of treatment. These include one doxorubicin cardiomyopathy, one myelodysplastic syndrome (MDS), and four cases of acute myelogenous leukemia (AML), all distributed without pattern among the four regimens.

Less than 2% of patients reported, late significant cardiac toxicity requiring treatment. The q 3 week regimens had a slight higher incidence of late cardiotoxicity than did the q 2 week regimens (2% vs. 1%, p=0.11) Severe post-chemotherapy neurotoxicity was rare overall but more frequent in the concurrent chemotherapy than in the sequential regimens (4% vs. 2%, P=0.0050). Fifty-eight patients have developed second primaries (Table 7), including eleven cases of AML or MDS (inclusive of deaths) diagnosed from 10 to 42 months after study entry, eighteen invasive breast cancers and three cases of ductal carcinoma in situ, all distributed without pattern among the four regimens. The 3-year incidence of AML or MDS was 0.18%. This is similar to a prior Intergroup trial (0.17%) for a similar patient population at the same median follow-up. Our incidence of leukemia was not influenced by filgrastim

### Discussion

Previous trials have shown that adding new, effective drugs sequentially into adjuvant treatment regimens can improve survival in early breast cancer. (Perloff M, Norton L, Korzun A H, et al: Postsurgical adjuvant chemotherapy of stage II breast carcinoma with or without crossover to a non-cross-resistant regimen: a Cancer and Leukemia Group B study. J Clin Oncol 14(5):1589-1598, May 1996) In addition, as predicted by theory, sequential chemotherapy has proven superior to a strictly alternating pattern. (Norton L. Implications of kinetic heterogeneity in clinical oncology. Semin Oncol 12:231-249, 1985) A recently reported trial of sequential A'C vs. concurrent AC in the adjuvant setting demonstrated no therapeutic differences, with more toxicity in the sequential arm, but there were by intention major differences between the arms in the dose levels of each drug. (Haskell C M, Green S J, Sledge G W Jr, et al: Phase III comparison of adjuvant high-dose doxorubicin plus cyclophosphamide (AC) versus sequential doxorubicin followed by cyclophosphamide (A->C) in breast cancer patients with 0-3 positive nodes (intergroup 0137). Proc Am Soc Clin Oncol 142, 2002 (abst)) Interpretation of this latter trial is complicated by considerations of dose-response, and the seeming lack of incremental benefit for A and C above certain dose thresholds. Yet the prospective, randomized comparison of sequential combination chemotherapy with concurrent combination chemotherapy using the same agents at the same dose levels and the same dose densities has never before been performed. In INT C9741 this comparison was accomplished by testing AC'T vs. A'T'C, with an additional manipulation of testing each schedule at two different dose densities, in a two-by-two factorial design.

At three years after completion of accrual, the total number of relapses was lower than anticipated at this protocol-specified analysis. We speculate that this may be related, in part, to greater use of tamoxifen in this trial compared to CALGB 8541 and possibly to a stage shift-within stage-due to improved mammographic screening. Nevertheless, the DFS has sufficiently matured at one and two years of follow-up that the statistically significant improvement due to dose density at one and two years will not be lost with further observation.

The DFS and OS advantages of dose density were not accompanied by an increase in toxicity. Indeed, the use of filgrastim in the dose dense regimens resulted in a statistically significant decrease in granulocyte toxicity. However, the low rate of hospitalization and the absence of mortality during chemotherapy illustrate the safety of all four treatment regimens. The low rate of neutropenic sepsis also supports the safety of using a baseline granulocyte count of 1000/μL (rather than the traditional 1500/μL) for administering chemotherapy. The use of the lower limit also may account for the infrequent treatment delays.

At present these data are consistent with mathematical predictions that dose density would improve therapeutic results and that sequential chemotherapy that maintains dose density would preserve efficacy while reducing toxicity. Several caveats are appropriate. The results might be drug and disease specific, the maximum follow-up of five years is still relatively short and treatment-related patterns of late recurrence (including local recurrence) and toxicity may yet emerge. Also, confidence in the OS benefits of a dose-dense schedule remains to be firmly established.

Filgrastim is an expensive intervention. Compared to standard treatment, it adds thousands of dollars to the chemotherapy regimen. Its cost/benefit ratio must be carefully considered. However, use of Filgrastim (G-CSF) as a daily treatment in the intervals between one or more of the treatments is an aspect of this invention.

The statistically significant OS benefits observed for the dose-dense regimens warrant further research to proceed in the context of these data. Oncologists may consider the implications of this study for clinical practice. This data set will continue to be followed using standard statistical methodology, and further reports will be generated.

Our results indicate interesting directions for further research. For example, sequential dose-dense single agent therapy could permit the rapid integration of new drugs into therapeutic regimens, including biological agents. Shorter inter-treatment intervals (i.e., beginning retreatment as soon as the granulocyte count reaches 1000/l, rather than at a fixed time interval) may also be used. Quality of life on such treatments might also be profitably explored. Furthermore, research into the biological etiology of Gompertzian growth and the molecular mechanisms of its perturbation could be used to hypothesize new, empirically verifiable dose-schedule manipulations.

TABLE 1
Patient characteristics and pretreatment variables
Seq 3 wks = Sequential A->T->C q 3 weeks
Concur 3 wks = Concurrent AC->T q 3 weeks
Seq 2 wks = Sequential A->T->C q 2 weeks
Concur 2 wks = Concurrent AC->T q 2 weeks
REGIMEM

| | | | |
|---|---|---|---|
| I | II | III Concur | IV Concur |
| Seq 3 wks | Seq 2 wks | 3 wks | 2 wks |
| N (%) | N (%) | N (%) | N (%) |

Characteristic

| | | | | |
|---|---|---|---|---|
| Total treated | 484 (100%) | 493 (100%) | 501 (100%) | 495 (100%) |

Stratificaion

| | | | | |
|---|---|---|---|---|
| # Positive | | | | |
| Nodes: | | | | |
| 1-3 | 287 (59%) | 292 (59%) | 301 (60%) | 293 (59%) |
| 4-9 | 139 (29%) | 143 (29%) | 142 (28%) | 145 (29%) |
| 10+ | 57 (12%) | 58 (12%) | 57 (11%) | 57 (11%) |
| Missing | 10 (2%) | 10 (2%) | 15 (3%) | 9 (2%) |

Demographics

| | | | | |
|---|---|---|---|---|
| Age: | | | | |
| <40 | 64 (13%) | 75 (15%) | 84 (17%) | 75 (15%) |
| 40-49 | 172 (36%) | 172 (35%) | 175 (35%) | 168 (34%) |
| 50-59 | 166 (34%) | 149 (30%) | 161 (32%) | 163 (33%) |
| 60-69 | 70 (14%) | 86 (17%) | 64 (13%) | 78 (16%) |
| 70+ | 12 (3%) | 11 (2%) | 17 (3%) | 11 (2%) |
| Race: | | | | |
| White | 402 (83%) | 407 (83%) | 398 (79%) | 416 (84%) |
| Hispanic | 22 (5%) | 21 (4%) | 18 (4%) | 19 (4%) |
| Black | 46 (10%) | 53 (11%) | 70 (14%) | 47 (10%) |
| Other | 14 (2%) | 12 (2%) | 15 (3%) | 13 (3%) |
| Menopausal | | | | |
| Status: | | | | |
| Pre- | 241 (50%) | 237 (48%) | 241 (49%) | 238 (48%) |
| Post- | 235 (48%) | 249 (51%) | 254 (50%) | 247 (50%) |
| Missing | 8 (2%) | 7 (1%) | 6 (1%) | 10 (2%) |
| ER Status: | | | | |
| Negative | 163 (34%) | 175 (35%) | 164 (33%) | 160 (32%) |
| Positive | 313 (64%) | 311 (63%) | 327 (65%) | 325 (66%) |
| Missing | 8 (2%) | 7 (2%) | 10 (2%) | 10 (2%) |
| Tumor Size: | | | | |
| ≦2 cm | 185 (38%) | 212 (43%) | 194 (39%) | 199 (40%) |
| >2 cm | 289 (60%) | 271 (55%) | 292 (58%) | 287 (58%) |
| Surgery: | | | | |
| Lumpectomy | 162 (33%) | 173 (35%) | 185 (37%) | 187 (37%) |
| Mastectomy | 312 (65%) | 306 (62%) | 300 (60%) | 301 (61%) |
| Other | 7 (1%) | 10 (2%) | 11 (2%) | 4 (1%) |
| Unknown | 3 (1%) | 4 (1%) | 5 (1%) | 3 (1%) |
| Menopausal/ | | | | |
| Tamox | | | | |
| Pre-/got tamox | 160 (66%) | 156 (66%) | 149 (62%) | 153 (64%) |
| Post-/got tamox | 173 (74%) | 189 (76%) | 186 (73%) | 192 (78%) |

TABLE 2
Site(s) of first relapse by regimen

| | Treatment Arm | | | |
|---|---|---|---|---|
| | Seq 3 wks | Seq 2 wks | Concur 3 wks | Concur 2 wks |
| | N (%) | N (%) | N (%) | N (%) |
| Total failures | 93 (100%) | 67 (100%) | 86 (100%) | 69 (100%) |
| Site of failure | | | | |
| Local only | 23 (25%) | 18 (27%) | 19 (22%) | 14 (20%) |
| Distant only | 58 (62%) | 44 (66%) | 56 (65%) | 46 (67%) |
| Local and | 12 (13%) | 5 (7%) | 11 (13%) | 9 (13%) |
| distant | | | | |
| concurrently | | | | |

TABLE 3a
Multivariate Cox Proportional Hazards Model:
Disease-Free Survival (N = 1973)

| | | | 95% Confidence | | |
|---|---|---|---|---|---|
| | Comparison | Risk | Interval | | |
| Variable | for RR* | Ratio | Lower | Upper | P-Value |
| No. of Positive | 10 vs. 1 | 2.20 | 1.75 | 2.77 | <0.0001 |
| Nodes** | | | | | |
| Tumor Size (cm)** | 5 vs. 2 | 1.53 | 1.27 | 1.85 | <0.0001 |
| Menopausal Status | Pre vs. Post | 1.08 | 0.85 | 1.35 | 0.54 |
| Estrogen Receptor | Neg vs. Pos | 3.30 | 2.62 | 4.17 | <0.0001 |
| Status | | | | | |
| Sequence | Seq vs. Con | 1.07 | 0.85 | 1.34 | 0.58 |
| Density | q 3 vs. q 2 wk | 1.35 | 1.07 | 1.70 | 0.010 |
| Interaction between | | | | | 0.40 |
| Density and | | | | | |
| Sequence | | | | | |

*First category names the group at higher risk of failure.
**A square root transformation was used in analyses.

TABLE 3b
Multivariate Cox Proportional Hazards Model:
Overall Survival (N = 1973)

| | | | 95% Confidence | | |
|---|---|---|---|---|---|
| | Comparison | Risk | Interval | | |
| Variable | for RR* | Ratio | Lower | Upper | P-Value |
| No. of Positive | 10 vs. 1 | 2.34 | 1.74 | 3.14 | <0.0001 |
| Nodes** | | | | | |
| Tumor Size (cm)** | 5 vs. 2 | 1.49 | 1.16 | 1.92 | 0.0019 |
| Menopausal Status | Pre vs. Post | 1.11 | 0.82 | 1.50 | 0.50 |
| Estrogen Receptor | Neg vs. Pos | 5.71 | 4.08 | 8.00 | <0.0001 |
| Status | | | | | |
| Sequence | Seq vs. Con | 1.12 | 0.83 | 1.51 | 0.48 |
| Density | q 3 vs. q 2 wk | 1.47 | 1.08 | 1.99 | 0.013 |
| Interaction between | | | | | 0.13 |
| Density and | | | | | |
| Sequence | | | | | |

*
*First category names the group at higher risk of failure.
**A square root transformation was used in analyses.

TABLE 4
Major Toxicities
Toxicity which occurred during protocol treatment.

| | Grade of Toxicity | | | | |
|---|---|---|---|---|---|
| | Arm | 3 (Sevr) | 4 (L.T.) | 5 (Leth) | Total |
| WBC | 1 | 2(0%) | 4(1%) | 0(0%) | 479 |
| | 2 | 0(0%) | 1(0%) | 0(0%) | 490 |
| | 3 | 3(1%) | 57(11%) | 0(0%) | 500 |
| | 4 | 1(0%) | 28(6%) | 0(0%) | 493 |
| Granulocytes/bands | 1 | 0(0%) | 113(24%) | 0(0%) | 479 |
| | 2 | 1(0%) | 14(3%) | 0(0%) | 490 |
| | 3 | 0(0%) | 214(43%) | 0(0%) | 500 |
| | 4 | 1(0%) | 46(9%) | 0(0%) | 493 |
| Nausea | 1 | 22(5%) | 1(0%) | 0(0%) | 479 |
| | 2 | 34(7%) | 1(0%) | 0(0%) | 490 |
| | 3 | 41(8%) | 3(1%) | 0(0%) | 500 |
| | 4 | 41(8%) | 0(0%) | 0(0%) | 493 |
| Vomiting | 1 | 10(2%) | 4(1%) | 0(0%) | 479 |
| | 2 | 14(3%) | 4(1%) | 0(0%) | 490 |
| | 3 | 32(6%) | 8(2%) | 0(0%) | 500 |
| | 4 | 18(4%) | 12(2%) | 0(0%) | 493 |
| Diarrhea | 1 | 5(1%) | 1(0%) | 0(0%) | 479 |
| | 2 | 8(2%) | 4(1%) | 0(0%) | 490 |
| | 3 | 7(1%) | 5(1%) | 0(0%) | 500 |
| | 4 | 5(1%) | 0(0%) | 0(0%) | 493 |
| Stomatitis | 1 | 5(1%) | 0(0%) | 0(0%) | 479 |
| | 2 | 4(1%) | 2(0%) | 0(0%) | 490 |
| | 3 | 14(3%) | 0(0%) | 0(0%) | 500 |
| | 4 | 9(2%) | 4(1%) | 0(0%) | 493 |
| Cardiac function | 1 | 5(1%) | 1(0%) | 0(0%) | 479 |
| | 2 | 4(1%) | 0(0%) | 0(0%) | 490 |
| | 3 | 1(0%) | 1(0%) | 0(0%) | 500 |
| | 4 | 0(0%) | 1(0%) | 0(0%) | 493 |
| Other cardiac | 1 | 2(0%) | 0(0%) | 0(0%) | 479 |
| | 2 | 0(0%) | 0(0%) | 0(0%) | 490 |
| | 3 | 0(0%) | 0(0%) | 0(0%) | 500 |
| | 4 | 1(0%) | 0(0%) | 0(0%) | 493 |
| Phlebitis/thrombosis | 1 | 3(1%) | 0(0%) | 0(0%) | 479 |
| | 2 | 4(1%) | 0(0%) | 0(0%) | 490 |
| | 3 | 3(1%) | 0(0%) | 0(0%) | 500 |
| | 4 | 4(1%) | 0(0%) | 0(0%) | 493 |
| Sensory | 1 | 21(4%) | 0(0%) | 0(0%) | 479 |
| | 2 | 19(4%) | 1(0%) | 0(0%) | 490 |
| | 3 | 25(5%) | 2(0%) | 0(0%) | 500 |
| | 4 | 19(4%) | 0(0%) | 0(0%) | 493 |
| Motor | 1 | 4(1%) | 0(0%) | 0(0%) | 479 |
| | 2 | 4(1%) | 0(0%) | 0(0%) | 490 |
| | 3 | 8(2%) | 1(0%) | 0(0%) | 500 |
| | 4 | 5(1%) | 0(0%) | 0(0%) | 493 |
| Pain | 1 | 19(4%) | 0(0%) | 0(0%) | 479 |
| | 2 | 33(7%) | 1(0%) | 0(0%) | 490 |
| | 3 | 31(6%) | 3(1%) | 0(0%) | 500 |
| | 4 | 46(9%) | 1(0%) | 0(0%) | 493 |
| Other neuro | 1 | 1(0%) | 0(0%) | 0(0%) | 479 |
| | 2 | 10(2%) | 1(0%) | 0(0%) | 490 |
| | 3 | 2(0%) | 0(0%) | 0(0%) | 500 |
| | 4 | 9(2%) | 1(0%) | 0(0%) | 493 |
| Skin | 1 | 8(2%) | 1(0%) | 0(0%) | 479 |
| | 2 | 15(3%) | 3(1%) | 0(0%) | 490 |
| | 3 | 2(0%) | 0(0%) | 0(0%) | 500 |
| | 4 | 11(2%) | 1(0%) | 0(0%) | 493 |
| Myalgias/arthralgias | 1 | 23(5%) | 0(0%) | 0(0%) | 479 |
| | 2 | 25(5%) | 0(0%) | 0(0%) | 490 |
| | 3 | 25(5%) | 2(0%) | 0(0%) | 500 |
| | 4 | 26(5%) | 0(0%) | 0(0%) | 493 |
| Infection | 1 | 14(3%) | 1(0%) | 0(0%) | 479 |
| | 2 | 19(4%) | 0(0%) | 0(0%) | 490 |
| | 3 | 27(5%) | 0(0%) | 0(0%) | 500 |
| | 4 | 13(3%) | 2(0%) | 0(0%) | 493 |

Note:
1 = Arm 1 (A → T → C q 3 weeks)
2 = Arm 2 (A → T → C q 2 weeks)
3 = Arm 3 (AC → T q 3 weeks)
4 = Arm 4 (AC → T q 2 weeks)

TABLE 5
Dose reductions
Seq 3 wks = Sequential A->T->C q 3 weeks
Concur 3 wks = Concurrent AC->T q 3 weeks
Seq 2 wks = Sequential A->T->C q 2 weeks
Concur 2 wks = Concurrent AC->T q 2 weeks

| | Treatment Arm | | | |
|---|---|---|---|---|
| | Seq 3 wks | Seq 2 wks | Concur 3 wks | Concur 2 wks |
| Dose reduction | N (%) | N (%) | N (%) | N (%) |
| Total with | 103 (100%) | 101 (100%) | 104 (100%) | 104 (100%) |
| Dose data | | | | |
| During Doxorubicin | 7 (7%) | 5 (5%) | 1 (1%) | 3 (3%) |
| During Cyclophosphamide | 1 (1%) | 3 (3%) | 5 (5%) | 5 (5%) |
| During Taxol | 1 (1%) | 7 (7%) | 4 (4%) | 5 (5%) |

TABLE 6
Treatment-related deaths
There have been six treatment-related deaths.

| Arm | Survival | COD |
|---|---|---|
| 1: Seq A→T→C q3 wks | 30 mos | Heart failure |
| 1: Seq A→T→C q3 wks | 40 mos | AML |
| 1: Seq A→T→C q3 wks | 41 mos | AML |
| 2: Seq A→T→C q2 wks | 23 mos | AML |
| 3: Concur AC→T q3 wks | 30 mos | MDS |
| 3: Concur AC→T q3 wks | 39 mos | Infection secondary to AML |

TABLE 7
Second Primaries

| | Treatment Arm | | | |
|---|---|---|---|---|
| | | | Concur | Concur |
| | Seq 3 wks | Seq 2 wks | 3 wks | 2 wks |
| | N (%) | N (%) | N (%) | N (%) |
| Total treated | 484 (100%) | 493 (100%) | 501 (100%) | 495 (100%) |
| Total w/sec | 16 (3%) | 16 (3%) | 12 (2%) | 14 (3%) |
| primary | | | | |
| Contralat breast | 9 | 2 | 6 | 1 |
| DCIS | 1 | 1 | 0 | 1 |
| Cervix | 1 | 0 | 0 | 1 |
| Ovary | 0 | 1 | 0 | 0 |
| Endometrium | 0 | 1 | 0 | 1 |
| AML/MDS | 2 | 3 | 4 | 2 |
| Basal/ | 0 | 3 | 1 | 2 |
| squamous | | | | |
| Melanoma | 1 | 1 | 0 | 1 |
| Lung | 0 | 2 | 1 | 0 |
| Thyroid | 0 | 0 | 0 | 2 |
| Colon | 0 | 0 | 0 | 1 |
| Intestine | 0 | 0 | 0 | 1 |
| Bladder | 0 | 0 | 0 | 1 |
| Renal | 2 | 0 | 0 | 0 |
| Pancreas | 0 | 1 | 0 | 0 |
| Pituitary | 0 | 1 | 0 | 0 |

What is claimed is:

1. A method for treating breast cancer in a patient having a chemotherapy responsive breast cancer, comprising the steps of:
(a) administering to the patient in a first plurality of chemotherapy cycles a therapeutically-effective and well-tolerated amount of doxorubicin, said first plurality chemotherapy cycles being administered in a dose-dense protocol;
(b) after the completion of the first plurality of chemotherapy cycles, administering to the patient in a second plurality of chemotherapy cycles a therapeutically-effective and well-tolerated amount of a taxane chemotherapy agent, said second plurality of chemotherapy cycles being administered in a dose-dense protocol; and
(c) after the completion of the second plurality of chemotherapy cycles, administering to the patient in a third plurality of chemotherapy cycles a therapeutically-effective and well-tolerated amount of cyclophosphamide, said third plurality of chemotherapy cycles being administered in a dose-dense protocol.

2. The method of claim 1, wherein the dose dense interval between treatments in the chemotherapy cycles about 14 days.

3. The method of claim 2, wherein the number of cycles in each plurality of chemotherapy cycles is 3 or more.

4. The method of claim 3, wherein the number of cycles in each plurality of chemotherapy cycles is 4.

5. The method of claim 4, wherein the doxorubicin is administered in an amount of 60 mg/m².

6. The method of claim 5, wherein the taxane is paclitaxel.

7. The method of claim 6, wherein the paclitaxel is administered in an amount of 175 mg/m².

8. The method of claim 7, wherein the cyclophosphamide is administered in an amount of 600 mg/².

9. The method of claim 1, wherein the number of cycles in each plurality of chemotherapy cycles is 3 or more.

10. The method of claim 9, wherein the number of cycles in each plurality of chemotherapy cycles is 4.

11. The method of claim 10, wherein the doxorubicin is administered in an amount of 60 mg/m².

12. The method of claim 11, wherein the taxane is paclitaxel.

13. The method of claim 12, wherein the paclitaxel is administered in an amount of 175 mg/m².

14. The method of claim 13, wherein the cyclophosphamide is administered in an amount of 600 mg/m².

15. The method of claim 1, wherein the doxorubicin is administered in an amount of 60 mg/m².

16. The method of claim 15, wherein the taxane is paclitaxel.

17. The method of claim 16, wherein the paclitaxel is administered in an amount of 175 mg/m².

18. The method of claim 17, wherein the cyclophosphamide is administered in an amount of 600 mg/m².

19. The method of claim 1, wherein the taxane is paclitaxel.

20. The method of claim 19, wherein the paclitaxel is administered in an amount of 175 mg/m².

21. The method of claim 20, wherein the cyclophosphamide is administered in an amount of 600 mg/m².

22. The method of claim 1, wherein the cyclophosphamide is administered in an amount of 600 mg/m².

23. The method of claim 1, further comprising the step of administering to the patient a therapeutically effective amount of granulocyte colony stimulating factor (G-CSF) during the intervals between treatments in one or more of the chemotherapy cycles.

24. The method of claim 23, wherein the G-CSF is administered in each interval between treatments in all of the chemotherapy cycles.

25. The method of claim 23, wherein the dose dense interval between treatments in the chemotherapy cycles about 14 days.

26. The method of claim 25, wherein the number of cycles in each plurality of chemotherapy cycles is 3 or more.

27. The method of claim 26, wherein the number of cycles in each plurality of chemotherapy cycles is 4.

28. The method of claim 27, wherein the doxorubicin is administered in an amount of 60 mg/m².

29. The method of claim 28, wherein the taxane is paclitaxel.

30. The method of claim 29, wherein the paclitaxel is administered in an amount of 175 mg/m².

31. The method of claim 30, wherein the cyclophosphamide is administered in an amount of 600 mg/m².

32. The method of claim 23, wherein the number of cycles in each plurality of chemotherapy cycles is 3 or more.

33. The method of claim 32, wherein the number of cycles in each plurality of chemotherapy cycles is 4.

34. The method of claim 33, wherein the doxorubicin is administered in an amount of 60 mg/m².

35. The method of claim 34, wherein the taxane is paclitaxel.

36. The method of claim 35, wherein the paclitaxel is administered in an amount of 175 mg/m².

37. The method of claim 36, wherein the cyclophosphamide is administered in an amount of 600 mg/m².

38. A method for providing improved chemotherapeutic efficacy in the treatment of a patient suffering from cancer, comprising the steps of:
(a) administering to a cancer patient in need of chemotherapeutic treatment a first plurality of chemotherapy cycles of a therapeutically-effective and well-tolerated amount of a first chemotherapy agent, said first chemotherapy agent being effective against the cancer, and said first plurality chemotherapy cycles being administered in a dose-dense protocol; and
(b) after the completion of the first plurality of chemotherapy cycles, administering to the patient in a second plurality of chemotherapy cycles of a therapeutically-effective and well-tolerated amount of a second chemotherapy agent, different from the first chemotherapy agent, said second plurality of chemotherapy cycles being administered in a dose-dense protocol, whereby the effectiveness of the first and second chemotherapy agents are enhanced as compared to treatment with the first and second chemotherapy agents in combination and in a non-dose-dense protocol.

39. In a method for providing chemotherapeutic treatment to a patient suffering from cancer, said method comprising administering to the patient one or more types of chemotherapeutic agent in a plurality of treatments, the improvement wherein the amount of each type of chemotherapeutic agent used is the optimal amount, and the treatments are given in a dose-dense protocol.
